(19) United States
(12) Patent Application Publication
(10) Pub. No.: US 2014/0012035 A1
(43) Pub. Date: Jan. 9, 2014

(54) Production of Substituted Phenylene Aromatic Diesters

(71) Applicants: Linfeng Chen, Missouri City, TX (US); Tak W. Leung, Houston, TX (US); Kuanqiang Gao, Pearland, TX (US); Tao Tao, Houston, TX (US)

(72) Inventors: Linfeng Chen, Missouri City, TX (US); Tak W. Leung, Houston, TX (US); Kuanqiang Gao, Pearland, TX (US); Tao Tao, Houston, TX (US)

(21) Appl. No.: 14/007,188

(22) PCT Filed: Mar. 27, 2012
(86) PCT No.: PCT/US12/30696

§ 371 Date: Sep. 24, 2013

Related U.S. Application Data
(60) Provisional application No. 61/468,928, filed on Mar. 29, 2011.

(51) Int. Cl. C07C 67/08

(52) U.S. Cl. CPC C07C 67/08
USPC 560/85

(57) ABSTRACT

Synthesis pathways for a precursor to 5-tert-butyl-3-methyl-1, 2-phenylene dibenzoate are provided. The precursor is methylcatechol and/or 5-tert-butyl-3-methylcatechol.

### BACKGROUND

The present disclosure relates to the production of substituted phenylene aromatic diesters.

Substituted phenylene aromatic diesters are used as internal electron donors in the preparation of procatalyst compositions for the production of olefin-based polymers. In particular, Ziegler-Natta catalysts containing 5-tert-butyl-3-methyl-1,2-phenylene dibenzoate as internal electron donor show high catalyst activity and high selectivity during polymerization. These catalysts produce olefin-based polymer (such as propylene-based polymer) with high isotacticity and broad molecular weight distribution.

Known is 5-tert-butyl-3-methylcatechol (or “BMC”) as a precursor for the production of 5-tert-butyl-3-methyl-1,2-phenylene dibenzoate (or “BMPD”). Commercial supply of BMC, however, is limited, unreliable, and difficult to obtain. The art therefore recognizes the need for additional sources and/or additional synthesis procedures for the reliable, consistent, efficient, and economical supply of BMC.

### SUMMARY

The present disclosure provides unique synthetic pathways for the production of 5-tert-butyl-3-methylcatechol or BMC. The processes disclosed herein are particularly advantageous for the commercial production of BMC because of the efficiencies (i.e., efficiencies in terms of energy, cost, time, productivity, and/or readily available starting reagents) provided thereby. The BMC can then be converted to BMPD via numerous synthetic pathways. Provision of reliable BMC advantageously simplifies production of BMPD thereby promoting production of olefin-based polymers with improved properties—vis-à-vis Ziegler-Natta olefin polymerization catalysts containing BMPD.

The present disclosure provides a process. In an embodiment, a process is provided and includes halogenating, under reaction conditions, o-cresol to form a halogenated methylphenol. The process includes hydrolyzing, under reaction conditions, the halogenated methylphenol to form 3-methylcatechol. The process includes alkylating, under reaction conditions, the 3-methylcatechol with a member selected from t-butanol, isobutylene, isobutyl halide, and t-butyl halide to form 5-t-butyl-3-methylcatechol. The process includes benzoylating, under reaction conditions, the 5-t-butyl-3-methylcatechol to form 5-t-butyl-3-methyl-1,2-phenylene dibenzoate.

The disclosure provides another process. In an embodiment, a process is provided and includes halogenating, under reaction conditions, o-cresol to form a halogenated methylphenol. The process includes alkylating, under reaction conditions, the halogenated methylphenol with a member selected from t-butanol, isobutylene, isobutyl halide, and t-butyl halide to form 2-halo-4-tert-butyl-6-methylphenol. The process includes hydrolyzing, under reaction conditions, the 2-halo-4-tert-butyl-6-methylphenol to form 5-t-butyl-3-methylcatechol. The process includes benzoylating, under reaction conditions, the 5-t-butyl-3-methylcatechol to form 5-t-butyl-3-methyl-1,2-phenylene dibenzoate.

The disclosure provides another process. In an embodiment, a process is provided and includes reacting an o-cresol, under reaction conditions, with an alcohol or an alkyl halide to form a 1-alkoxy-2-methylbenzene. The process includes halogenating, under reaction conditions, the 1-alkoxy-2-methylbenzene to form a halogenated 1-alkoxy-2-methylbenzene. The process includes first hydrolyzing, under reaction conditions, the halogenated 1-alkoxy-2-methylbenzene to form a 2-alkoxy-3-methylphenol. The process includes alkylating, under reaction conditions, the 2-alkoxy-3-methylphenol to form 5-tert-butyl-1,2-dialkoxy-3-methylbenzene. The process includes second hydrolyzing, under reaction conditions, the 5-tert-butyl-1,2-dialkoxy-3-methylbenzene to form 5-t-butyl-3-methylcatechol. The process includes benzoylating, under reaction conditions, the 5-t-butyl-3-methylcatechol to form 5-t-butyl-3-methyl-1,2-phenylene dibenzoate.

The disclosure provides another process. In an embodiment, a process is provided and includes formylating, under reaction conditions, catechol to form 2,3-dihydroxybenzaldehyde. The process includes hydrogenolyzing, under reaction conditions, 2,3-dihydroxybenzaldehyde to form 3-methyl-catechol. The process includes alkylating, under reaction conditions, the 3-methyl-catechol to form 5-t-butyl-3-methylcatechol. The process includes benzoylating, under reaction conditions, the 5-t-butyl-3-methylcatechol to form 5-t-butyl-3-methyl-1,2-phenylene dibenzoate.

The disclosure provides another process. In an embodiment, a process is provided and includes hydrogenolyzing, under reaction conditions, o-vanillin to form 2-methoxy-6-methylphenol. The process includes hydrolyzing, under reaction conditions, the 2-methoxy-6-methylphenol and forming 3-methylcatechol.

An advantage of the present disclosure is the production of BMC and/or BMPD by way of readily available and/or common starting material(s).

An advantage of the present disclosure is an improved process for the production of substituted phenylene aromatic diester, such as 5-tert-butyl-3-methyl-1,2-phenylene dibenzoate.

An advantage of the present disclosure is the production of a precursor to BMC/BMPD, namely, methylcatechol.

An advantage of the present disclosure is the provision of a precursor to 5-tert-butyl-3-methyl 1,2-phenylene dibenzoate, namely, 5-tert-butyl-3-methylcatechol.

An advantage of the present disclosure is the provision of a plurality of synthesis pathways to produce 5-tert-butyl-3-methylcatechol.

An advantage of the present disclosure is the production of 5-tert-butyl-3-methyl-1,2-phenylene dibenzoate using inexpensive starting materials.

An advantage of the present disclosure is numerous synthesis pathways for the production of substituted phenylene aromatic diester, such as 5-tert-butyl-3-methyl-1,2-phenylene dibenzoate, thereby ensuring a reliable supply of same for the production of propylene-based polymers.

An advantage of the present disclosure is a process for large scale production of substituted phenylene aromatic diester.

An advantage of the present disclosure is an environmentally-safe, non-toxic production process for substituted phenylene aromatic diester.

An advantage of the present disclosure is the large scale production of substituted phenylene aromatic diester.

An advantage of the present disclosure is a simple, time-effective, and/or cost-effective purification process for substituted phenylene aromatic diester.

### BRIEF DESCRIPTION OF THE DRAWINGS

FIG. 1 is a reaction scheme in accordance with an embodiment of the present disclosure.

FIG. 2 is a reaction scheme in accordance with an embodiment of the present disclosure.

FIG. 3 is a reaction scheme in accordance with an embodiment of the present disclosure.

FIG. 4 is a reaction scheme in accordance with an embodiment of the present disclosure.

FIG. 5 is a reaction scheme in accordance with an embodiment of the present disclosure.

FIG. 6 is a schematic representation of a polymerization system in accordance with an embodiment of the present disclosure.

### DETAILED DESCRIPTION

The present disclosure is directed to the production of substituted phenylene aromatic diester. The compound 5-tert-butyl-3-methylcatechol (or “BMC”) is found to be an effective precursor for the production of the substituted phenylene aromatic diester, 5-tert-butyl-3-methyl-1,2-phenylene dibenzoate (or “BMPD”). BMPD is an effective internal electron donor in Ziegler-Natta catalysts. The processes disclosed herein advantageously provide economical (time, energy, productivity, and/or starting reagent economies), simplified, up-scalable, synthesis pathways to BMC with yields acceptable for commercial/industrial application thereof. Reliable production of BMC correspondingly contributes to reliable and economical production of 5-tert-butyl-3-methyl-1,2-phenylene dibenzoate (BMPD), which in turn contributes to the production of olefin-based polymer (propylene-based polymer in particular) with improved properties.

1. BMC/BMPD from o-cresol via Direct Halogenation

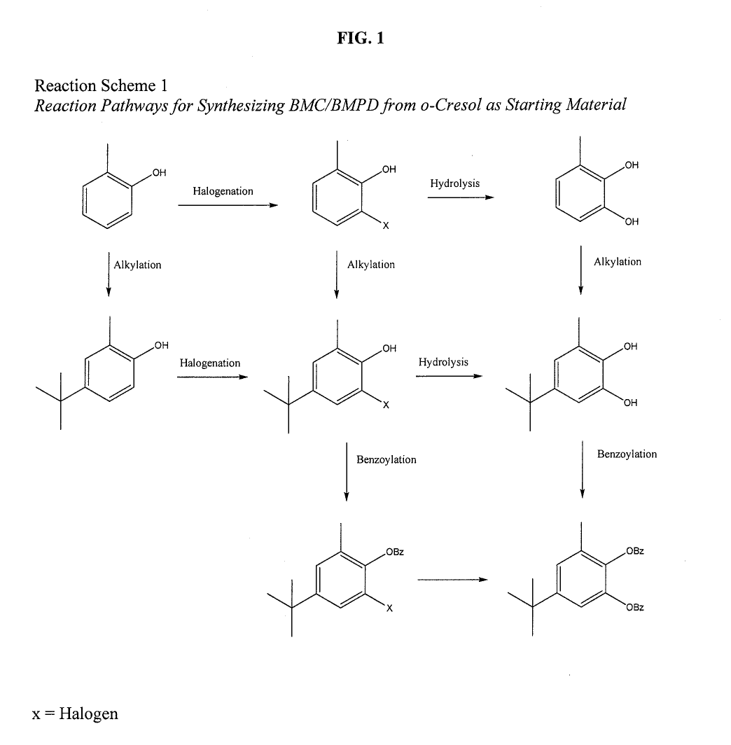

In an embodiment, BMC and/or BMPD are/is produced from ortho-cresol (hereafter o-cresol). Use of o-cresol as a starting material is advantageous because o-cresol is readily available from numerous sources. The o-cresol may or may not include substituents. BMC and/or BMPD are/is made from o-cresol via subsequent halogenation, hydrolysis, alkylation, and benzoylation in any order and as shown in Reaction Scheme 1 of FIG. 1.

The o-cresol may be halogenated into 2-halo-6-methylphenol, hydrolyzed into 3-methylcatechol, alkylated into BMC, and benzoylated into BMPD. Alternatively, the o-cresol may be halogenated into 2-halo-6-methylphenol, alkylated into 2-halo-4-tert-butyl-6-methylphenol, hydrolyzed into BMC, and benzoylated into BMPD. Each of these steps occurs under reaction conditions. As used herein, “reaction conditions,” are temperature, pressure, reactant concentrations, solvent selection, reactant mixing/addition parameters, and/or other conditions within a reaction vessel that promote reaction between the reagents and formation of the resultant product.

The term “halogenating,” or “halogenation,” or “halogenation reaction,” is the introduction of a halogen radical into an organic compound. Halogenation occurs by way of reaction with a halogenating agent. Nonlimiting examples of suitable halogenating agents include elemental halogens (F₂, Cl₂, Br₂, I₂), boron trihalides (such as boron tri-bromide), N-bromosuccinimide (NBS), a brominating agent, and/or N-chlorosuccinimide (NCS), a chlorinating agent.

The term “alkylating,” or “alkylation,” or “alkylation reaction” is the introduction of an alkyl radical into an organic compound. An “organic compound” is a chemical compound that contains a carbon atom.

The term “benzoylating,” or “benzoylation,” “or benzoylation reaction” as used herein, is a chemical reaction whereby a benzoyl group is attached to an organic compound. In an embodiment, the benzoylation involves reacting an organic compound with benzoyl halide, benzoic acid, and/or benzoic anhydride, optionally in the presence of a base, such as pyridine and/or triethylamine.

As used herein, “hydrolyzing,” or “hydrolysis,” or “hydrolysis reaction” is a chemical reaction whereby a hydroxyl group replaces a functional group. In an embodiment, the hydrolysis reaction is catalyzed by a base (such as NaOH) and/or a salt, such as such as copper (II) sulfate.

The present disclosure provides a process. In an embodiment, a process is provided and includes halogenating, under reaction conditions, o-cresol to form a halogenated methylphenol. The halogenated methylphenol is hydrolyzed, under reaction conditions, to form 3-methylcatechol. The process further includes alkylating, under reaction conditions, the 3-methylcatechol with a member selected from t-butanol, isobutylene, isobutyl halide, and t-butyl halide (and any combination thereof) to form 5-t-butyl-3-methylcatechol. The 5-t-butyl-3-methylcatechol is benzoylated, under reaction conditions, to form 5-t-butyl-3-methyl-1,2-phenylene dibenzoate.

The process utilizes o-cresol as a starting material. The o-cresol is halogenated, under reaction conditions, to form a halogenated methylphenol (or halo-methylphenol). The halogenating agent may be any halogenating agent as disclosed above.

In an embodiment, the halogenation occurs by way of bromination. A brominating agent is reacted with the o-cresol under reaction conditions to form 2-bromo-6-methylphenol. Nonlimiting examples of suitable brominating agents are elemental bromine, boron tribromide, and N-bromosuccinimide.

The process further includes hydrolyzing, under reaction conditions, the halo-methylphenol to form 3-methylcatechol. In an embodiment, 2-bromo-6-methylphenol is hydrolyzed, the hydrolysis reaction catalyzed by a base (such as NaOH) and/or a salt, such as such as copper (II) sulfate.

The process includes alkylating, under reaction conditions, the 3-methylcatechol with t-butanol, isobutylene, isobutyl halide, and/or t-butyl halide (and any combination thereof). This reaction forms 5-t-butyl-3-methylcatechol (BMC). In an embodiment, alkylation occurs with the addition of an inorganic acid (such as sulfuric acid) or a Lewis acid (such as aluminum trichloride) to a mixture of the 3-methylcatechol and the tert-butanol in heptane to form 5-t-butyl-3-methylcatechol (BMC).

The process includes benzoylating, under reaction conditions, the 5-t-butyl-3-methylcatechol to form 5-t-butyl-3-methyl-1,2-phenylene dibenzoate (BMPD). In an embodiment, benzoylation proceeds by reacting BMC with benzoyl chloride in the presence of a base under reaction conditions, and forming BMPD. Nonlimiting examples of suitable base include pyridine, triethylamine, trimethylamine, and/or molecular sieves.

In an embodiment, 4-t-butyl-2-methylphenol, which can be synthesized via alkylation of o-cresol, is utilized as the starting material for production of BMC/BMPD as shown in FIG. 1. The 4-t-butyl-2-methylphenol is halogenated to form 2-halo-4-tert-butyl-6-methylphenol. In a further embodiment, 2-halo-4-tert-butyl-6-methylphenol is hydrolyzed to form BMC, and subsequently benzoylated to form BMPD. The halogenation, hydrolysis and/or benzoylation of the 4-t-butyl-2-methylphenol may be performed in the same manner as when o-cresol is used as the starting material and as disclosed above. In another embodiment, 2-halo-4-tert-butyl-6-methylphenol is benzoylated into 2-halo-4-tert-butyl-6-methylphenyl benzoate, and then the halo group is substituted to form BMPD.

The disclosure provides another process. In an embodiment, a process is provided and includes halogenating, under reaction conditions, o-cresol to form a halogenated methylphenol. The halogenated methylphenol is alkylated, under reaction conditions, with t-butanol, isobutylene, isobutyl halide, and/or t-butyl halide (and any combination thereof) to form 2-halo-4-tert-butyl-6-methylphenol. The 2-halo-4-tert-butyl-6-methylphenol is hydrolyzed, under reaction conditions, to form 5-t-butyl-3-methylcatechol. The process includes benzoylating, under reaction conditions, the 5-t-butyl-3-methylcatechol to form 5-t-butyl-3-methyl-1,2-phenylene dibenzoate.

In an embodiment, halogenation occurs by way of bromination. The process includes brominating the o-cresol, under reaction conditions, to form 2-bromo-6-methylphenol.

The foregoing processes using an o-cresol for BMC/BMPD production as the starting material are depicted in Reaction Scheme 1 as shown in FIG. 1.

2. o-cresol as Starting Material Via Ether Protection

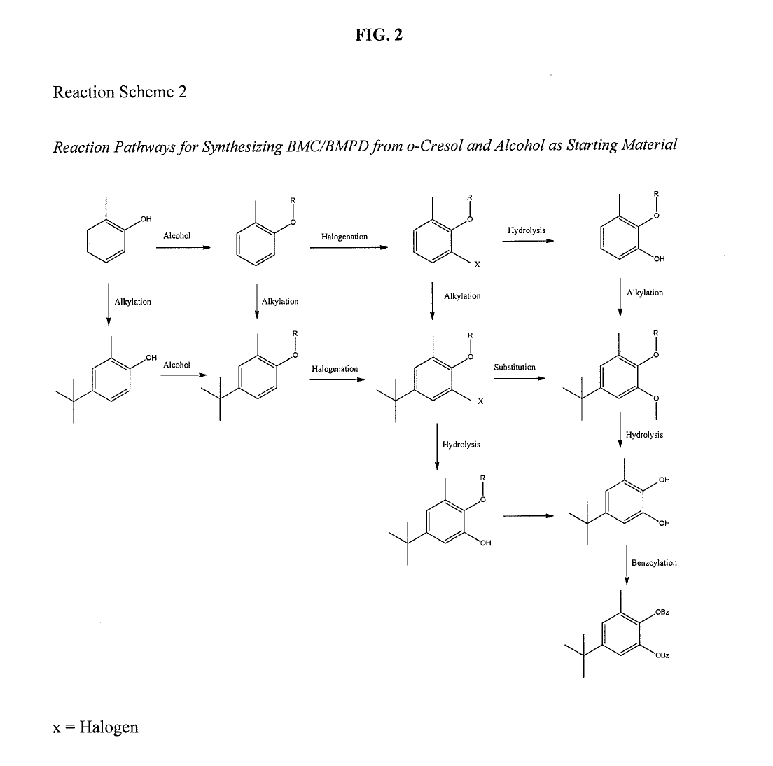

In embodiment, BMC and/or BMPD are/is produced using o-cresol via protection of the hydroxyl group by formation of an ether from reaction with an alcohol or alkyl halide. The o-cresol may or may not include substituents. BMC and/or BMPD are/is made from o-cresol via subsequent ether protection, halogenation, hydrolysis, alkylation, and benzoylation in any order and as shown in Reaction Scheme 2 of FIG. 2.

The disclosure provides another process. In an embodiment, a process is provided and includes reacting an o-cresol, under reaction conditions, with an alcohol or alkyl halide to form a 1-alkoxy-2-methylbenzene. The 1-alkoxy-2-methylbenzene is halogenated, under reaction conditions, to form a halogenated 1-alkoxy-2-methylbenzene. The process further includes first hydrolyzing, under reaction conditions, the halogenated 1-alkoxy-2-methylbenzene to form a 2-alkoxy-3-methylphenol. The 2-alkoxy-3-methylphenol is alkylated, under reaction conditions, to form 5-tert-butyl-1,2-dialkoxy-3-methylbenzene. The process includes second hydrolyzing, under reaction conditions, the 5-tert-butyl-1,2-dialkoxy-3-methylbenzene to form 5-t-butyl-3-methylcatechol. The 5-t-butyl-3-methylcatechol is benzoylated, under reaction conditions, to form 5-t-butyl-3-methyl-1,2-phenylene dibenzoate.

In an embodiment, the alcohol is selected from methanol and/or ethanol.

In an embodiment, the process includes catalyzing the o-cresol and alcohol reaction with an acid. Nonlimiting examples of suitable acids for catalysis include sulfuric acid and/or hydrochloric acid.

In an embodiment, the process includes catalyzing the second hydrolyzing with an acid. Nonlimiting examples of suitable acids for hydrolysis catalysis include inorganic acids such as boron trichloride and/or sulfuric acid.

In an embodiment, the process includes brominating the 1-alkoxy-2-methylbenzene to form 1-bromo-2-alkoxy-3-methylbenzene.

The foregoing processes using an o-cresol and an alcohol as starting material for BMC/BMPD production are depicted in Reaction Scheme 2 as shown in FIG. 2.

### 3. Formylation Reaction Scheme

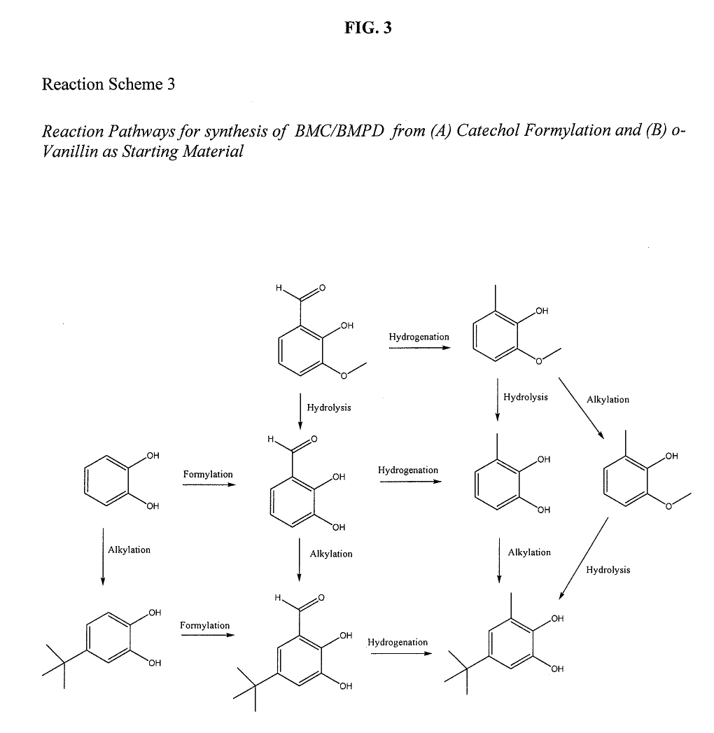

In embodiment, BMC and/or BMPD are/is produced using catechol as a starting material and formylating the catechol. The catechol may or may not include substituents. BMC and/or BMPD are/is made from catechol via formylation, hydrogenation, and alkylation in any order as shown in Reaction Scheme 3 in FIG. 3.

The disclosure provides another process. In an embodiment, a process is provided and includes formylating, under reaction conditions, catechol to form 2,3-dihydroxybenzaldehyde. The 2,3-dihydroxybenzaldehyde is hydrogenolyzed, under reaction conditions, to form 3-methylcatechol. The process includes alkylating, under reaction conditions, the 3-methylcatechol to form 5-t-butyl-3-methylcatechol. The 5-t-butyl-3-methylcatechol is benzoylated, under reaction conditions, to form 5-t-butyl-3-methyl-1,2-phenylene dibenzoate. The term “hydrogenolyzing,” or “hydrogenolysis,” or “hydrogenolysis reaction” is a chemical reaction whereby a carbon-carbon or carbon-heteroatom single bond is cleaved by hydrogen. Nonlimiting examples of suitable hydrogenolyzing agents include catalytic hydrogenolyzing agents (such as palladium catalysts) and borohydrides, such as sodium cyano-borohydride.

In an embodiment, the process includes catalyzing the formylation reaction with magnesium chloride.

In an embodiment, the hydrogenolyzation reaction includes reacting the 2,3-dihydroxybenzaldehyde with hydrogen and/or hydrazine.

The foregoing processes which formylate the starting material catechol to produce BMC/BMPD are depicted in Reaction Scheme 3 as shown in FIG. 3.

4. o-Vanillin Starting Material

In an embodiment, 3-methylcatechol is produced using ortho-vanillin (hereafter o-vanillin) as a starting material. The 3-methylcatechol may be subsequently used to produce BMC and/or BMPD. Use of o-vanillin as starting material is advantageous because o-vanillin is readily available from numerous sources. The o-vanillin may or may not include substituents.

The process for producing 3-methylcatechol from o-vanillin may include providing o-vanillin as a starting material and hydrogenolyzing, hydrolyzing, and alkylating, in any order; the o-vanillin to form o-vanillin reaction intermediates. The hydrogenolyzation, hydrolysis and/or alkylation reactions form the o-vanillin and its subsequent reaction intermediates into 3-methylcatechol.

The disclosure provides another process. In an embodiment, a process is provided and includes hydrogenolyzing, under reaction conditions, o-vanillin to form 2-methoxy-6-methylphenol. The 2-methoxy-6-methylphenol is hydrolyzed, under reaction conditions, to form 3-methylcatechol.

In an embodiment, the process includes alkylating, under reaction conditions, the 3-methylcatechol with t-butanol, isobutylene, isobutyl halide, and/or t-butyl halide to form 5-t-butyl-3-methylcatechol.

The disclosure provides another process. In an embodiment, a process is provided and includes hydrogenolyzing, under reaction conditions, o-vanillin to form 2-methoxy-6-methylphenol. The 2-methoxy-6-methylphenol is alkylated, under reaction conditions, to form 4-tert-butyl-2-methyl-6-methoxyphenol. 4-tert-butyl-2-methyl-6-methoxyphenol is then hydrolyzed, under reaction conditions, to form 5-t-butyl-3-methylcatechol.

The foregoing processes which use o-vanillin as the starting material to produce 3-methylcatechol are depicted in Reaction Scheme 3 as shown in FIG. 3.

5. 1,2-dialkoxybenzene Intermediates

The disclosure provides another process wherein the hydroxyl groups in catechol are protected by conversion into ether groups, a 1,2-dialkoxybenzene intermediate. In an embodiment, a process is provided and includes alkylating, under reaction conditions, a 1,2-dialkoxy-4-t-butyl-benzene, which can be obtained from alkylating o-cresol and then reacting with an alcohol, to form 1,2-dialkoxy-4-t-butyl-6-methyl-benzene. In a further embodiment, the alkylation is accomplished via treating 1,2-dialkoxy-4-t-butyl-benzene with an alkyllithium followed by reaction with a methyl halide. The process further includes hydrolyzing, under reaction conditions, the 1,2-dialkoxy-4-t-butyl-6-methyl-benzene to form 5-t-butyl-3-methylcatechol.

In an embodiment, the 1,2-dialkoxy-4-t-butyl-benzene is 1,2 dimethoxy-4-t-butyl-benzene.

In an embodiment, the process includes methylating 4-t-butyl-catechol, under reaction conditions, to form the 1,2 dimethoxy-4-t-butyl-benzene.

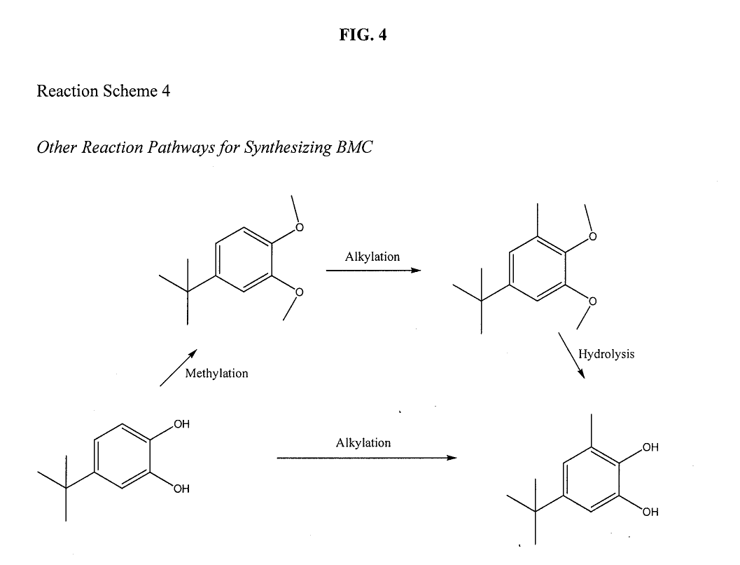

The foregoing processes with 1,2-dialkoxy-4-t-butyl-benzene as the reaction intermediate are depicted in Reaction Scheme 4 in FIG. 4.

### 5. Direct Oxidation

The disclosure provides another process wherein 5-t-butyl-3-methylcatechol is synthesized from o-cresol by alkylation and then oxidation in any order.

In an embodiment, the process includes alkylating o-cresol with t-butanol, isobutylene, isobutyl halide, and/or t-butyl halide to form 4-tert-butyl-2-methylphenol. The process further includes oxidizing 4-tert-butyl-2-methylphenol to form 5-t-butyl-3-methylcatechol.

In an embodiment, the process includes oxidizing o-cresol to form 3-methylcatechol. The process further includes alkylating 3-methylcatechol to form 5-t-butyl-3-methylcatechol.

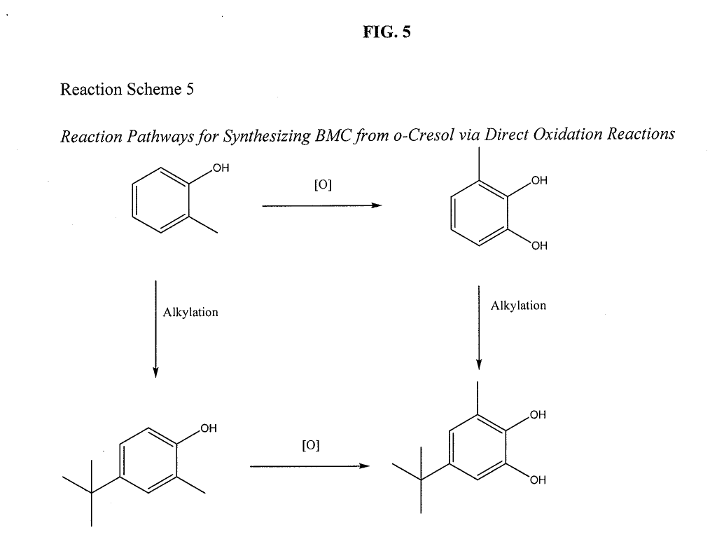

The foregoing processes with o-cresol as starting material via alkylation and oxidation are depicted in Reaction Scheme 5 in FIG. 5.

The BMPD is advantageously applied as an internal electron donor in procatalyst/catalyst compositions for the production of olefin-based polymers (propylene-based polymers in particular) as disclosed in U.S. provisional application No. 61/141,902 filed on Dec. 31, 2008 and U.S. provisional application No. 61/141,959 filed on Dec. 31, 2008, the entire content of each application incorporated by reference herein.

In an embodiment, a catalyst composition is provided. As used herein, “a catalyst composition” is a composition that forms an olefin-based polymer when contacted with an olefin under polymerization conditions. The catalyst composition includes a procatalyst composition, and a cocatalyst. The procatalyst composition is a combination of a magnesium moiety, a titanium moiety and an external electron donor containing a substituted phenylene aromatic diester, such as BMPD. The BMPD is produced by way of any process disclosed herein. The catalyst composition may optionally include an external electron donor and/or an activity limiting agent.

In an embodiment, a process for producing an olefin-based polymer is provided. The process includes contacting an olefin with the catalyst composition under polymerization conditions. The catalyst composition includes a substituted phenylene aromatic diester, such as BMPD. The substituted phenylene aromatic diester can be any substituted phenylene dibenzoate as disclosed herein. The process further includes forming an olefin-based polymer, such as an ethylene-based polymer and a propylene-based polymer.

As used herein, “polymerization conditions” are temperature and pressure parameters within a polymerization reactor suitable for promoting polymerization between the catalyst composition and an olefin to form the desired polymer. The polymerization process may be a gas phase, a slurry, or a bulk polymerization process, operating in one, or more than one, reactor.

In an embodiment, polymerization occurs by way of condensed mode gas phase polymerization. As used herein, “condensed mode gas phase polymerization” is the passage of an ascending fluidizing medium, the fluidizing medium containing one or more monomers, in the presence of a catalyst through a fluidized bed of polymer particles maintained in a fluidized state by the fluidizing medium. “Fluidization,” “fluidized,” or “fluidizing” is a gas-solid contacting process in which a bed of finely divided polymer particles is lifted and agitated by a rising stream of gas. Fluidization occurs in a bed of particulates when an upward flow of fluid through the interstices of the bed of particles attains a pressure differential and frictional resistance increment exceeding particulate weight. Thus, a “fluidized bed” is a plurality of polymer particles suspended in a fluidized state by a stream of a fluidizing medium. A “fluidizing medium” is one or more olefin gases, optionally a carrier gas (such as H₂ or N₂) and optionally a liquid (such as a hydrocarbon) which ascends through the gas-phase reactor.

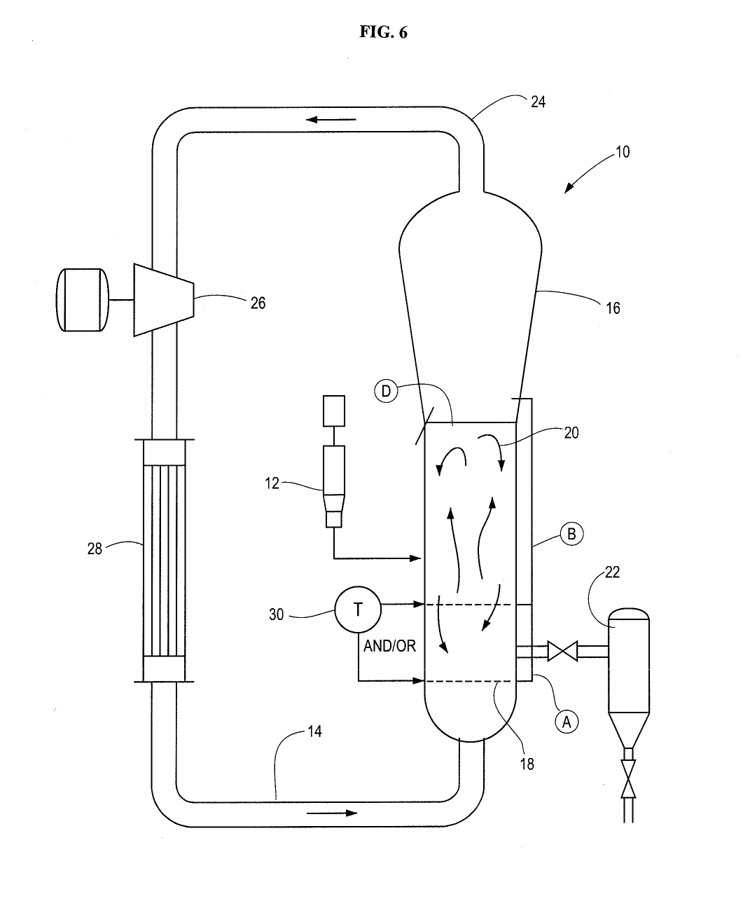

FIG. 6 shows a condensed-mode gas-phase polymerization reactor 10 which includes a recycle stream, where a catalyst 12 and monomer feed 14 enter a gas phase reactor 16 and are swept above a distributor plate 18 into the fluidized bed mixing zone 20. The monomer is polymerized into polymer that is then withdrawn via a discharge apparatus 22. At the same time a recycle stream 24 is withdrawn from the reactor 16 and passed to a compressor 26. The reactor 16 has a diameter D. From the compressor 26, the recycle stream is passed to a heat exchanger 28, and thereafter the recycle stream is passed back into the reactor along with the monomer feed 14. Fluid is formed by cooling the recycle stream below the dew point temperature. An inert liquid (such as an induced cooling agent) may be introduced into the recycle stream to increase the dew point temperature of the recycle stream. A condensed mode process is advantageous because it has the ability to remove greater quantities of heat generated by polymerization thereby increasing the polymer production capacity of a fluidized bed polymerization reactor.

Condensed mode gas phase polymerization is a three phase system composed of liquid, gas and solids.

It has been discovered that condensed liquid accumulates in the bottom half of the reactor. During production, (especially when such reactors are run at high throughput or production rates) the amount of condensed liquid entering the reactor significantly increases because of the increased cooling demand. The accumulation of the condensed liquid in the bottom portion of the reactor leads to numerous operational problems, including higher liquid content of the removed polymer product, reduced overall catalyst yields, product inconsistency, and instabilities in reactor behavior, including fluidization, temperature control and continuity. Conventional responses to the problem of accumulation liquid such as increasing fluidization velocity and/or increasing bed temperature are ineffective.

It has been found that this accumulation of condensed liquid is the result of a dynamic transition which includes a profile of temperature bands that are present above the distributor plate 18. As shown in FIG. 6, the profile of temperature bands includes a cold, wet band A in the bottom portion (typically the bottom third portion) of the reactor and a warm drier band B in the top portion (typically the top two-thirds portion of the reactor). Reactor temperature probes in conventional reactors are located in the warm band. It has been found that provision of the temperature probe in the warm band is not effective in controlling the temperature in the cold wet band.

It has been discovered that placement of one or more temperature probes 30 at a location from 0.5 D (D being the diameter of the reactor) to 1.5 D above the distribution plate 18 advantageously places the temperature probe 30: (i) at the transition between temperature bands A and B and/or (ii) in the cold wet temperature band A. Placement of the temperature probe in this manner allows effective control of the cold wet band A and the capability to remove or avoid this band.

Placement of the temperature probe 30 at 0.5 D to 1.5 D above the distributor plate 18 enables the gas phase polymerization reactor 10 to produce polyolefin at greater production rates and/or greater space-time yield without the accumulation of condensed liquid in the cold band A of the reactor. The advantages of placing the temperature probe 30 at 0.5 D-1.5 D above the distribution plate 18 are as follows.

(1) Higher catalyst productivity and lower conversion costs. When accumulation of the liquid occurs, the liquid can account for about one-third of measured bed weight. This means that actual catalyst residence time is reduced, resulting in reduced catalyst productivity. Removing liquid from the bed increases productivity significantly.

(2) Significant increases in production rates. Accumulated liquid near the bottom of the reactor causes excessive liquid being carried with polymer product into the product discharge system (PDS) (i.e., discharge apparatus 22). The result is low temperatures and high peak pressures in the PDS, which is a safety issue and limits production rates because vent recovery becomes overloaded. Placement of the temperature probe at 0.5 D-1.5 D reduces/eliminates accumulated liquid thereby reducing/eliminating the liquid present in the polymer product and reducing/eliminating safety risk with the discharge system.

(3) Removal of the liquid from the reactor bottom lowers monomer usage (TMR) at high rates through lower losses in vent recovery.

### DEFINITIONS

All references to the Periodic Table of the Elements herein shall refer to the Periodic Table of the Elements, published and copyrighted by CRC Press, Inc., 2003. Also, any references to a Group or Groups shall be to the Groups or Groups reflected in this Periodic Table of the Elements using the IUPAC system for numbering groups. Unless stated to the contrary, implicit from the context, or customary in the art, all parts and percents are based on weight. For purposes of United States patent practice, the contents of any patent, patent application, or publication referenced herein are hereby incorporated by reference in their entirety (or the equivalent US version thereof is so incorporated by reference), especially with respect to the disclosure of synthetic techniques, definitions (to the extent not inconsistent with any definitions provided herein) and general knowledge in the art.

Any numerical range recited herein, includes all values from the lower value to the upper value, in increments of one unit, provided that there is a separation of at least 2 units between any lower value and any higher value. As an example, if it is stated that the amount of a component, or a value of a compositional or a physical property, such as, for example, amount of a blend component, softening temperature, melt index, etc., is between 1 and 100, it is intended that all individual values, such as, 1, 2, 3, etc., and all subranges, such as, 1 to 20, 55 to 70, 197 to 100, etc., are expressly enumerated in this specification. For values which are less than one, one unit is considered to be 0.0001, 0.001, 0.01 or 0.1, as appropriate. These are only examples of what is specifically intended, and all possible combinations of numerical values between the lowest value and the highest value enumerated, are to be considered to be expressly stated in this application. In other words, any numerical range recited herein includes any value or subrange within the stated range.

The term “alkyl,” as used herein, refers to a branched or unbranched, saturated or unsaturated acyclic hydrocarbon radical. Nonlimiting examples of suitable alkyl radicals include, for example, methyl, ethyl, n-propyl, i-propyl, 2-propenyl (or allyl), vinyl, n-butyl, t-butyl, i-butyl (or 2-methylpropyl), etc. The alkyls have 1 and 20 carbon atoms.

The term “aryl,” as used herein, refers to an aromatic substituent which may be a single aromatic ring or multiple aromatic rings which are fused together, linked covalently, or linked to a common group such as a methylene or ethylene moiety. The aromatic ring(s) may include phenyl, naphthyl, anthracenyl, and biphenyl, among others. The aryls have 1 and 20 carbon atoms.

The term “composition,” as used herein, includes a mixture of materials which comprise the composition, as well as reaction products and decomposition products formed from the materials of the composition.

The term “comprising,” and derivatives thereof, is not intended to exclude the presence of any additional component, step or procedure, whether or not the same is disclosed herein. In order to avoid any doubt, all compositions claimed herein through use of the term “comprising” may include any additional additive, adjuvant, or compound whether polymeric or otherwise, unless stated to the contrary. In contrast, the term, “consisting essentially of” excludes from the scope of any succeeding recitation any other component, step or procedure, excepting those that are not essential to operability. The term “consisting of” excludes any component, step or procedure not specifically delineated or listed. The term “or”, unless stated otherwise, refers to the listed members individually as well as in any combination.

The term “ethylene-based polymer,” as used herein, is a polymer that comprises a majority weight percent polymerized ethylene monomer (based on the total amount of polymerizable monomers), and optionally may comprise at least one polymerized comonomer.

The term “olefin-based polymer” is a polymer containing, in polymerized form, a majority weight percent of an olefin, for example ethylene or propylene, based on the total weight of the polymer. Nonlimiting examples of olefin-based polymers include ethylene-based polymers and propylene-based polymers.

The term “polymer” is a macromolecular compound prepared by polymerizing monomers of the same or different type. “Polymer” includes homopolymers, copolymers, terpolymers, interpolymers, and so on. The term “interpolymer” means a polymer prepared by the polymerization of at least two types of monomers or comonomers. It includes, but is not limited to, copolymers (which usually refers to polymers prepared from two different types of monomers or comonomers, terpolymers (which usually refers to polymers prepared from three different types of monomers or comonomers), tetrapolymers (which usually refers to polymers prepared from four different types of monomers or comonomers), and the like.

The term, “propylene-based polymer,” as used herein, is a polymer that comprises a majority weight percent polymerized propylene monomer (based on the total amount of polymerizable monomers), and optionally may comprise at least one polymerized comonomer.

The term “substituted alkyl,” as used herein, refers to an alkyl as just described in which one or more hydrogen atom bound to any carbon of the alkyl is replaced by another group such as a halogen, aryl, substituted aryl, cycloalkyl, substituted cycloalkyl, heterocycloalkyl, substituted heterocycloalkyl, halogen, haloalkyl, hydroxy, amino, phosphido, alkoxy, amino, thio, nitro, and combinations thereof. Suitable substituted alkyls include, for example, benzyl, trifluoromethyl and the like.

The term “substituted phenylene aromatic diester” includes substituted 1,2-phenylene aromatic diester, substituted 1,3-phenylene aromatic diester, and substituted 1,4-phenylene aromatic diester. In an embodiment, the substituted phenylene diester is a 1,2-phenylene aromatic diester with the structure (A) below:

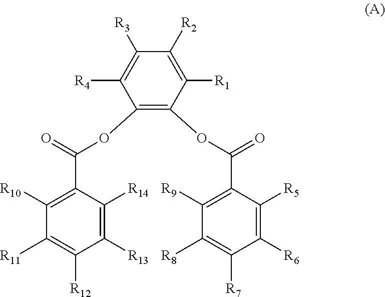

wherein R₁-R₁₄ are the same or different. Each of R₁-R₁₄ is selected from a hydrogen, substituted hydrocarbyl group having 1 to 20 carbon atoms, an unsubstituted hydrocarbyl group having 1 to 20 carbon atoms, an alkoxy group having 1 to 20 carbon atoms, a heteroatom, and combinations thereof. At least one of R₁-R₁₄ is not hydrogen.

### Test Methods

¹H nuclear magnetic resonance (NMR) data is obtained via a Bruker 400 MHz spectrometer in CDCl₃ (in ppm).

By way of example, and not limitation, examples of the present disclosure are provided.

### EXAMPLES

### Preparation of 2-methoxy-6-methylphenol from hydrogenation of o-vanillin

This reaction is performed inside a drybox for safety precautions because hydrogen gas is used. During the procedure, the drybox is purged periodically with nitrogen to ensure no build up of hydrogen gas. An adaptor with a balloon on one end is attached to a 250 mL flask with a side arm and a magnetic stir bar. One gram of Pd on carbon (5% Pd) is charged slowly into the flask. Then, 7.6 g of o-vanillin and 100 ml of methanol are added. Through the side-arm, hydrogen gas is introduced into the flask system until the balloon is inflated to a volume of about 250 ml. The reaction is allowed to stir at room temperature for 3 days. Hydrogen gas is added when the balloon deflates due to the reaction and diffusion. GC samples are taken to monitor the reaction. When reaction is complete, as evidenced by the appearance of the intermediate first and then by the appearance of the product, the gas inside the balloon and flask is released slowly. The reaction is stirred openly inside the dry box for another 10 minutes to ensure complete dissipation of hydrogen inside the flask into the dry box. The dry box is also purged several times with nitrogen. The flask is taken out of the drybox. The reaction mixture is filtered to separate off the catalyst. The solvent is removed to yield the crude product. The GC and NMR data are compared with the authentic sample to be 2-methoxy-6-methylphenol. Yield is 7.3 g or 95%.

### Preparation of 3-methylcatechol from hydrogenation of 2,3-dihydroxybenzaldehyde

The procedure is similar to that described for the hydrogenation of o-vanillin. The yield of this reaction by GC was 95%.

### Preparation of 3-methylcatechol from 2-methoxy-6-methylcatechol

To a 250 ml of flask 2-methoxy-6-methylphenol (5.0 g, 36.2 mmol) is charged along with 40 ml of a 48% aqueous hydrobromic acid solution. The mixture is heated to 85-90° C. for 6 hours. After cooling to room temperature, the mixture was extracted with ethyl acetate. The ethyl acetate extract is washed with water and brine, and then dried over magnesium sulfate. After filtration, the filtrate is concentrated, and dried in vacuo to yield 4.1 g (91.3%) of the product as a yellowish liquid. ¹H NMR: 6.71 (s, 3H), 5.20 (br.s, 2H), 2.25 (s, 3H).

### Preparation of 5-tert-butyl-2,3-dihydroxybenzaldehyde from 4-tert-butylcatechol

A 1-L 3-neck flask, equipped with stirrer, reflux condenser, thermometer, nitrogen inlet and bubbler is charged with 4-tert-butylcatechol (8.3 g, 50 mmol), and anhydrous acetonitrile (500 mL). To the solution is added triethylamine (24.9 mL, 3.75 equiv.), followed by paraformaldehyde (9.4 g, 313 mmol, 6.25 equiv.). Then anhydrous magnesium chloride (14.3 g, 150 mmol, 3 equiv.) is added slowly in small portions. The mixture is heated to reflux for 4 hours. After cooling to room temperature, 10% HCl (200 mL) is added and the mixture is stirred for 30 minutes. The mixture is then extracted with ether (5×100 mL). The combined ether extracts are washed with brine and dried over MgSO₄. After removal of solvent under vacuum, the residue is dried in vacuo to yield 3.1 g (30%). ¹H NMR: 10.91 (s, 1H, CHO), 9.91 (s, 1H, OH), 7.12 (s, 1H, ArH), 6.94 (s, 1H, ArH), 1.32 (s, 9H).

### Preparation of 4-tert-butyl-2-methyl-6-methoxyphenol from 2-methyl-6-methoxypehnol via a Friedel-Craft reaction

A 250 ml of flask is charged with 2-methoxy-6-methylcatechol (5.0 g, 36.2 mmol), ethylene dichloride (30 mL). To the stirred solution is added anhydrous aluminum chloride (0.72 g, 5.4 mmol, 0.15 equiv.), followed by the drop-wise addition of a solution of 2-chloro-2-methylpropane (4.4 ml, 39.8 mmol, 1.1 equiv.) in ethylene dichloride (30 mL). The mixture is stirred overnight, and then quenched with 1N HCl. After separation, the aqueous layer is extracted with ether. The combined organic solution is washed with brine, and dried over magnesium sulfate. After filtration, the filtrate is concentrated and dried in vacuo to yield 6.3 g (96.6%) of the product as an off-white solid. ¹H NMR: 6.75 (s, 2H), 5.56 (s, 1H), 3.87 (s, 3H), 2.25 (s, 3H), 1.29 (s, 9H).

It is specifically intended that the present disclosure not be limited to the embodiments and illustrations contained herein, but include modified forms of those embodiments including portions of the embodiments and combinations of elements of different embodiments as come within the scope of the following claims.

1. A process comprising:
halogenating, under reaction conditions, o-cresol to form a halogenated methylphenol;
hydrolyzing, under reaction conditions, the halogenated methylphenol to form 3-methylcatechol;
alkylating, under reaction conditions, the 3-methylcatechol with a member selected from the group consisting of t-butanol, isobutylene, isobutyl halide, and t-butyl halide to form 5-t-butyl-3-methylcatechol; and
benzoylating, under reaction conditions, the 5-t-butyl-3-methylcatechol to form 5-t-butyl-3-methyl-1,2-phenylene dibenzoate.

2. The process of claim 1 comprising brominating the o-cresol under reaction conditions to form 2-bromo-6-methylphenol.

3. A process comprising:
halogenating, under reaction conditions, o-cresol to form a halogenated methylphenol;
alkylating, under reaction conditions, the halogenated methylphenol with a member selected from the group consisting of t-butanol, isobutylene, isobutyl halide, and t-butyl halide to form 2-halo-4-tert-butyl-6-methylphenol;
hydrolyzing, under reaction conditions, the 2-halo-4-tert-butyl-6-methylphenol to form 5-t-butyl-3-methylcatechol; and
benzoylating, under reaction conditions, the 5-t-butyl-3-methylcatechol to form 5-t-butyl-3-methyl-1,2-phenylene dibenzoate.

4. The process of claim 3 comprising brominating the ortho-cresol under reaction conditions to form 2-bromo-6-methylphenol.

5-8. (canceled)

9. A process comprising:
formylating, under reaction conditions, catechol to form 2,3-dihydroxybenzaldehyde;
hydrogenolyzing, under reaction conditions, 2,3-dihydroxybenzaldehyde to form 3-methyl-catechol;
alkylating, under reaction conditions, the 3-methyl-catechol to form 5-t-butyl-3-methylcatechol; and
benzoylating, under reaction conditions, the 5-t-butyl-3-methylcatechol to form 5-t-butyl-3-methyl-1,2-phenylene dibenzoate.

10. The process of claim 9 comprising catalyzing, with magnesium chloride, the formylating.

11. The process of claim 9 wherein the hydrogenolyzing comprises reacting the 2,3-dihydroxybenzaldehyde with a member selected from the group consisting of hydrogen and hydrazine.

12-13. (canceled)
